(19) United States
(12) Patent Application Publication
(10) Pub. No.: US 2004/0067172 A1
(43) Pub. Date: Apr. 8, 2004

(54) Air freshener device with child resistant features

(75) Inventors: Michael Ehrlich, Fort Lee, NJ (US); Tina Jenuleson, Bergenfield, NJ (US)

Correspondence Address: Richard R. Muccino, 758 Springfield Avenue, Summit, NJ, 07901, US

(73) Assignee: Homescents Inc., Fort Lee, NJ

(21) Appl. No.: 10/265,947

(22) Filed: Oct. 7, 2002

(51) Int. Cl. A61L 9/04; A24F 25/00

(52) U.S. Cl. 422/123; 422/125; 239/40

(57) ABSTRACT

The present invention is directed to air freshener devices with child resistant features. The air freshener devices comprise (a) a housing having a socket portion; (b) a container for holding a liquid to be evaporated by heat, the container having a body for storing the liquid and a neck connected to the body, the neck engaging the socket portion of the housing thereby supporting the container on the housing, the neck having a passage therethrough; (c) a retaining ring fixed in the passage of the neck of the container; (d) a wick having an upper portion extending through the retaining ring and a lower portion extending into the body of the container, the lower portion of the wick absorbing the liquid from the container and moving the liquid from the lower portion into the upper portion of the wick by capillary action; and (e) heating means in the housing for heating the upper portion of the wick above the neck of the body to evaporate the liquid from the wick. The retaining ring is crimped by an amount which precludes upward extraction of the wick from the retaining ring.

### FIELD OF THE INVENTION

The present invention relates to air freshener devices which use wicks. More specifically, the present invention relates to air freshener devices which employ child-resistant wick retaining structures, reverse thread features, and a compact structure.

### DESCRIPTION OF THE BACKGROUND

Wick-based air freshener devices are known in the art for dispersing the vapor of liquids into the air. Such devices are often used in the home with liquids varying from insect repellents to air fresheners. Typically, in such devices, one end of a wick is partially submerged in the liquid to be dispersed. The liquid is contained in any suitable container. The partially submerged portion of the wick absorbs the liquid, some of which diffuses by capillary or wicking action into the exposed, unsubmerged portion of the wick. The exposed portion of the wick is locally heated, often by means of a ring-shaped heater which fits over the wick. This causes the liquid which has diffused into the exposed portion of the wick to evaporate into the surrounding air. Continual application of heat to the exposed portion of the wick results in an evaporation/absorption process that continues until the liquid is consumed.

A problem with conventional wick-based air freshener devices, as with many products suitable for home use, is the potential that a child will come into contact with the liquid contents of the device. Many of the liquids utilized with such devices can be harmful if swallowed, and some are harmful if merely touched. Therefore, it is desirable to make these devices child-resistant to reduce the chance that child will access the potentially harmful contents of the device.

U.S. Pat. No. 3,724,756 (Maltenfort) discloses a wick holder and storage insert for engagement in the neck opening of a container of liquid to be dispensed by vaporization. The wick holder and storage insert comprise a generally cup-shaped member having a generally tubular-body having a bottom wall provided with a slot. A generally non-stiffened wick extends through the slot above and below the bottom wall. The wick is dimensioned so as to extend outwardly from the cupshaped member in a use position. The periphery of the slot engages, compresses, and seals the wick whereby the wick in wetted condition is normally nonretractable through the slot. The cup-shaped insert is dimensioned so as to have an interior volume sufficient to accommodate the portion of the wick above the bottom wall.

U.S. Pat. No. 3,844,342 (Eninger et al.) discloses a heat pipe having a condenser region and an evaporator region and containing a working liquid. The heat pipe is provided with an artery of substantial length for transporting the working liquid between the condenser and evaporator regions forming at least one venting pore in the artery. The artery is constituted at least in part by a thin-wall portion having a pore of capillary size formed therein. The wall portion is so thin as to keep the pore open through the action of meniscus coalescence of any miniscule amount of the working liquid tending to fill the pore when the pore borders a gas bubble within the artery. The action of meniscus coalescence permits the pore to remain open for venting noncondensible gas that may be present within the artery during the priming thereof and until such time that the artery is fully primed with the working liquid.

U.S. Pat. No. 4,739,928 (O'Neil) discloses an air freshener dispenser comprising a container having an opening. An emanator pad holding means is detachably connected to the container and retains an emanator pad in contact with a wick. The pad holding means has a fragrance diffusion channel and an upwardly extending side wall member. A cover member has a top and a substantially cylindrical side wall and includes a fragrance diffusion aperture registrable with the fragrance diffusion channel. A retention means comprises a first mating surface associated with the pad holding means and a second mating surface associated with the cover member. The mating surface is in an operative position detachably retaining the cover member on the pad holding means. The first mating surface is inoperative in cooperation with the cover member substantially cylindrical side wall to detachably retain the cover member on the pad holding means. The cover member is rotatable in the detachably retained position with respect to the pad holding means. The pad holding means is nonrotatable with respect to the container by the torquing force for rotating the cover member on the pad holding means. The rotation of the cover member enables the fragrance diffusion aperture and the fragrance diffusion channel to be brought into registry.

U.S. Pat. No. 4,742,960 (Bustillo et al.) discloses a wick dispenser for volatile liquids which includes a bottle with a bottom and a mouth. An insert cup in the mouth has an opening in the bottom of the cup. A wick is folded to define a bight traversing the opening and engaging the wick at the outer extremity of the bight. The improvement comprises spaced cross bars on the wick carrier. One of the bars engages the inside of the bight. The other bars are adjacent to the outside of the bight. Stop means on the wick carrier limits the extension of the wick carrier and of the wick to a preselected extended position from the bottle. A different cross bar on the wick carrier is located between the folded portions of the wick and is engageable with the folded portions to urge them against the structure of the opening when the stop means engages the underside of the insert cup whereby closure of the opening is assisted.

U.S. Pat. No. 4,819,719 (Grote et al.) discloses an enhanced capillary assisted evaporative surface comprising a conductive substrate and several grooves located in the conductive substrate. The grooves have a generally rectangular shape with a generally radiused, necked-down opening. Land between the groves connects the grooves. A dendritic surface is included on the lands and the generally radiused, necked-down openings of the grooves.

U.S. Pat. No. 5,484,086 (Pu) discloses a perfume gas generating device including a container for the perfume. The container has a main body, a neck portion formed on an upper end of the main body, and a reservoir defined by the main body. A cap member is associated with the neck portion of the container and has an opening. A fiber-made wick member is disposed in the reservoir of the container. The wick member serves as an upward moving path of the perfume in the reservoir and has a top end extending outside the neck portion of the container. A heating system includes a heating mechanism disposed above the wick member and has a heat input end and a heat output end and a heat source controlling circuit for conducting heat to the heating mechanism. The heating mechanism forms a high temperature area above the top end of the wick member. The perfume is able to move upward along the wick member into the high temperature area to be vaporized into gas which dissipates into the ambient environment. The heating mechanism includes a transistor connected with the input end of the heating mechanism, a diode and a current-limiting resistor connected with the transistor, and an oscillating circuit having a variable resistor. The oscillating circuit is connected with the transistor through the current-limiting resistor and diode.

U.S. Pat. No. 5,647,053 (Schroeder et al.) discloses a vapor dispensing device which comprises an outer shell, a one piece electric plug heater block, and a wick. The one piece electric plug heater block has electric plug pins. The block is rotatably attached to the outer shell such that the block can rotate through a predefined range of rotation around an axis parallel to the plug pins. The wick is in fluid communication with a material to be dispensed and extending into an opening through the block. The opening has a shape defined by rotating a solid geometric shape transverse to the axis through an angle at least equal to the predefined range of rotation of the block. The block has an electric heating element in close proximity to the opening.

U.S. Pat. No. 5,669,767 (Bureau et al.) discloses a device for use in an oil lamp having an inside and a lip, to allow the diffusion of the scent of a perfume added to the oil. The lip lies in a generally horizontal plane. The device comprises a ringshaped element having several inwardly downwardly protruding tabs that are spaced apart to define gaps permitting uninterrupted air flow between the inside of the bottle and the ambient air. The tabs have free ends defining an opening and sized and shaped to receive and hold a wick supporting tube. The tube has a longitudinal axis perpendicular to the plane and an upper end from which a wick projects. The tube is inserted into the opening so that the upper end is held in place at a given longitudinal distance from the lip. When the device is placed on the lip and the wick is lit, heat generated by the lighting heats the tube and the oil and causes the perfume to be released into the ambient air through the gaps.

U.S. Pat. No. 5,725,152 (Akyu) discloses an air freshener dispenser comprising a container having a neck with an opening for containing a fragrant liquid composition. A wick depends downwardly into the container supported by a wick holder. A head assembly is monolithically molded and includes a hollow cylindrical member suitable for mating the head assembly with the neck of the container. An exterior ornamental part extends from the outermost of the cylindrical member. Several retaining means are formed on the top of the cylindrical member to extend uprightly therefrom and are disposed circumstantially at a predetermined interval to surround an opening matching with the container opening. An emanator medium of an absorbent material is removably retained in the head assembly and has a bottom part and ambient parts. A fixing member substantially comprises a shaft and a flat bottom, and is vertically inserted into an inner space defined by the retaining means of the head assembly for connecting the emanator medium to the head assembly, thereby holding the bottom part of the emanator medium in fluid contact with the top of the wick as well as causing the ambient parts of the emanator medium to stand upright.

U.S. Pat. No. 5,909,845 (Greatbatch et al.) discloses a child-resistant, wick-based liquid emanation system. The system comprises a container with an opening for holding a liquid. A wick is partially disposed within the container and has an extended portion extending through the opening of the container. A hollow overcap encases the extended portion of the wick. The overcap has a closed tip, separable from the overcap, and an open base. The base is attached to the container to cover the opening, the overcap closely conforming to the shape and size of the wick. The system is activated by separating the tip from the overcap.

U.S. Pat. No. 6,104,867 (Stathakis et al.) discloses a vapor dispensing device comprising a refill bottle unit including a wick in fluid communication with a material to be dispensed contained within the refill bottle and extending into a heater unit contained within a housing unit. The refill bottle unit and the housing unit include a guidance system to guide the refill bottle unit into the housing unit and to stabilize the bottle unit with respect to the housing unit. The center of the wick is relative to the heating unit when the refill bottle unit and the housing unit are being interconnected.

U.S. Pat. No. 6,236,807 (Ruffolo et al.) discloses a vapor emanation system comprising a housing having a socket portion. A container for a liquid to be evaporated by heat has a body for storing the liquid and a neck connected to the body and for engaging the socket portion of the housing for supporting the container on the housing. The neck has a passage there through. A retaining ring is fixed in the passage and has a hole there through. A wick has an upper portion extending through the hole of the retaining ring, and a lower portion extending down into the body of the container for absorbing liquid from the container and for moving the liquid into the upper portion of the wick by capillary action. A heating means in the housing heats the upper portion of the wick above the neck to evaporate liquid from the wick. A retaining pin extends through the wick below the neck and beyond the wick by an amount which precludes upward extraction of the wick from the retaining ring.

U.S. Pat. No. 6,354,513 (Basaganas Millan) discloses a plug for containers of evaporable liquids of the type comprising an annular body with a neck for coupling and permanent attachment to the corresponding neck of the container to which it is applied. The plug is provided with an axial concentric passage for placing a cylindrical wick, which is partially submerged in the liquid of the container and partially emerges outwards to allow evaporation through it of the liquid of the container. The plug is characterized in that the innermost or lowermost passage area of the wick comprises a concentric neck provided with slits along the main generatrix line. The slits define sectors which press on the lateral surface of the wick with the neck enveloped by a second neck of greater diameter which is also provided with slits along the main generatrix line so that the second neck is shorter than the neck. The property includes the sectors of the neck which press on the wick which are provided with triangular semi-pyramidal projections which provide means to allow an inwards displacement of the wick and prevent its displacement in the opposite direction. These properties are in order to prevent it from being separated from the plug once mounted on it, and preventing refilling of the container on which the plug is applied.

PCT application no. WO 98/46284 (Martin '284) discloses an air freshener dispenser device which is adapted for engagement and support by a wall electrical outlet, and which is an assembly of structural units. The air freshener dispenser device comprises a disposable cartridge having an elongated thermoplastic hollow body configuration with a sealed internal reservoir chamber of liquid air freshener medium. The upper end of the cartridge has a narrow stem extension of the internal chamber. An elongated wick extends from the chamber bottom up to the top of the stem extension. Integrally structured means are adapted for removal of a top portion of the cartridge stem extension to expose an upper section of the wick to the atmosphere. A heater means is detachably secured and positioned proximate to the cartridge stem extension for promotion of an freshener wicking into the atmosphere.

PCT application no. WO 98/46285 (Martin '285) discloses a disposable air freshener dispenser device which is adapted for engagement and support by a wall electrical outlet and which is an assembly of structural units. The disposable air freshener dispenser device comprises a cartridge having a thermoplastic tray having side walls with an upper edge which forms a peripheral margin around the open space of the tray. The flange has an elongate extension from one wall, and the surface of the elongate flange extension has a shallow flat recess extending the wall edge. A thin emanating absorbent matrix with an upper section fits within the flange flat recess, and with a lower section which extends across the open space of the tray to the opposite wall edge. A volatile liquid air freshener medium is contained within the tray interior. A first vapor-impermeable membrane covers the open space of the tray and the lower section of the absorbent matrix and is bonded to the tray peripheral margin and forms a sealed air freshener reservoir enclosure within the tray interior. A peelable second vapor-impermeable membrane covers the upper section of the absorbent matrix. An electrical-resistance type heating means is detachably secured and positioned proximate to the back surface of the tray elongated flange extension and the upper section of the absorbent. A molded plastic electrical plug housing with a flat front surface and two inlet openings is located within the plug housing. A detachable thin panel section is juxtapositioned on the flat front surface of the plug housing. The panel section comprises an electrical-resistance heater module and has two apertures corresponding in position to the inlet openings within the plug housing. A pair of metal prongs are positioned within the apertures of the panel section and the inlet openings of the plug housing and extend rearwardly from the plug housing for engagement with a wall electrical outlet. The prongs are adapted to conduct electric current to the electrical resistance heater module.

### IN THE FIGURES

FIG. 1 is a schematic drawing of the air freshener device of the present invention in a preferred embodiment.

FIG. 2 is a schematic drawing of three preferred embodiments of the invention illustrating the container, retaining ring, and wick of the air freshener device. In FIG. 2A, the higher cylindrical portion of the retaining ring is crimped forming a pressure point on the wick. In FIG. 2B, the lower cylindrical portion of the retaining ring is crimped forming a pressure point on the wick. In FIG. 2C, both the higher and the lower cylindrical portions of the retaining ring are crimped forming pressure points on the wick.

FIG. 3 is a schematic drawing comparing a top view of a conventional retaining ring for an air freshener device versus the retaining ring of the present invention. FIG. 3A is a schematic drawing of a top view of a conventional retaining ring for an air freshener device. FIG. 3B is a schematic drawing of a top view of the retaining ring of the present invention for an air freshener device.

### SUMMARY OF THE INVENTION

The present invention pertains to an air freshener device comprising:

(a) a housing having a socket portion;

(b) a container for holding a liquid to be evaporated by heat, the container having a body for storing the liquid and a neck connected to the body, the neck engaging the socket portion of the housing thereby supporting the container on the housing, the neck having a passage therethrough;

(c) a retaining ring fixed in the passage of the neck of the container;

(d) a wick having an upper portion extending through the retaining ring and a lower portion extending into the body of the container, the lower portion of the wick absorbing the liquid from the container and moving the liquid from the lower portion into the upper portion of the wick by capillary action; and

(e) heating means in the housing for heating the upper portion of the wick above the neck of the body to evaporate the liquid from the wick;

wherein the retaining ring is crimped by an amount which precludes upward extraction of the wick from the retaining ring.

The present invention also pertains to an air freshener device comprising:

(a) a housing having a socket portion;

(b) a container for holding a liquid to be evaporated by heat, the container having a body for storing the liquid and a neck connected to the body, the neck engaging the socket portion of the housing with reverse screw threads thereby supporting the container on the housing, the neck having a passage therethrough;

(c) a retaining ring fixed in the passage of the neck of the container, the retaining ring having a higher cylindrical portion which forms a flange on the top of the neck of the container, and which engages the container by press fit, and a lower cylindrical portion;

(d) a wick having an upper portion extending through the retaining ring and a lower portion extending into the body of the container, the lower portion of the wick absorbing the liquid from the container and moving the liquid from the lower portion into the upper portion of the wick by capillary action; and

(e) heating means in the housing for heating the upper portion of the wick above the neck of the body to evaporate the liquid from the wick, the heating means including a plug portion and electric blade means for being plugged into an electric wall socket for powering the heating means and for supporting the device on the wall;

wherein the retaining ring is crimped by an amount which precludes upward extraction of the wick from the retaining ring.

### DETAILED DESCRIPTION OF THE INVENTION

The present invention is directed to air freshener devices. The devices comprise (a) a housing having a socket portion; (b) a container for holding a liquid to be evaporated by heat, the container having a body for storing the liquid and a neck connected to the body, the neck engaging the socket portion of the housing thereby supporting the container on the housing, the neck having a passage therethrough; (c) a retaining ring fixed in the passage of the neck of the container; (d) a wick having an upper portion extending through the retaining ring and a lower portion extending into the body of the container, the lower portion of the wick absorbing the liquid from the container and moving the liquid from the lower portion into the upper portion of the wick by capillary action; and (e) heating means in the housing for heating the upper portion of the wick above the neck of the body to evaporate the liquid from the wick. The retaining ring is crimped by an amount which precludes upward extraction of the wick from the retaining ring.

The invention will be better understood from the following detailed description of the preferred embodiments taken in conjunction with the Figures, in which like elements are represented by like referenced numerals.

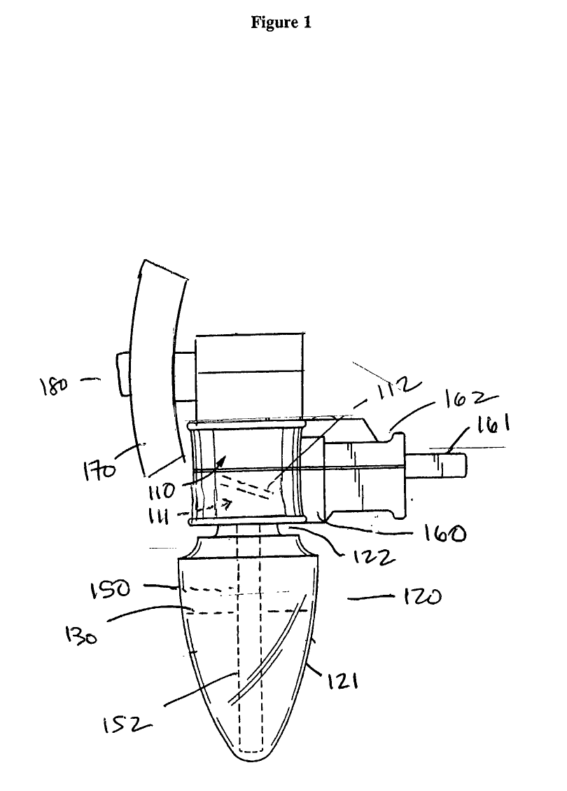

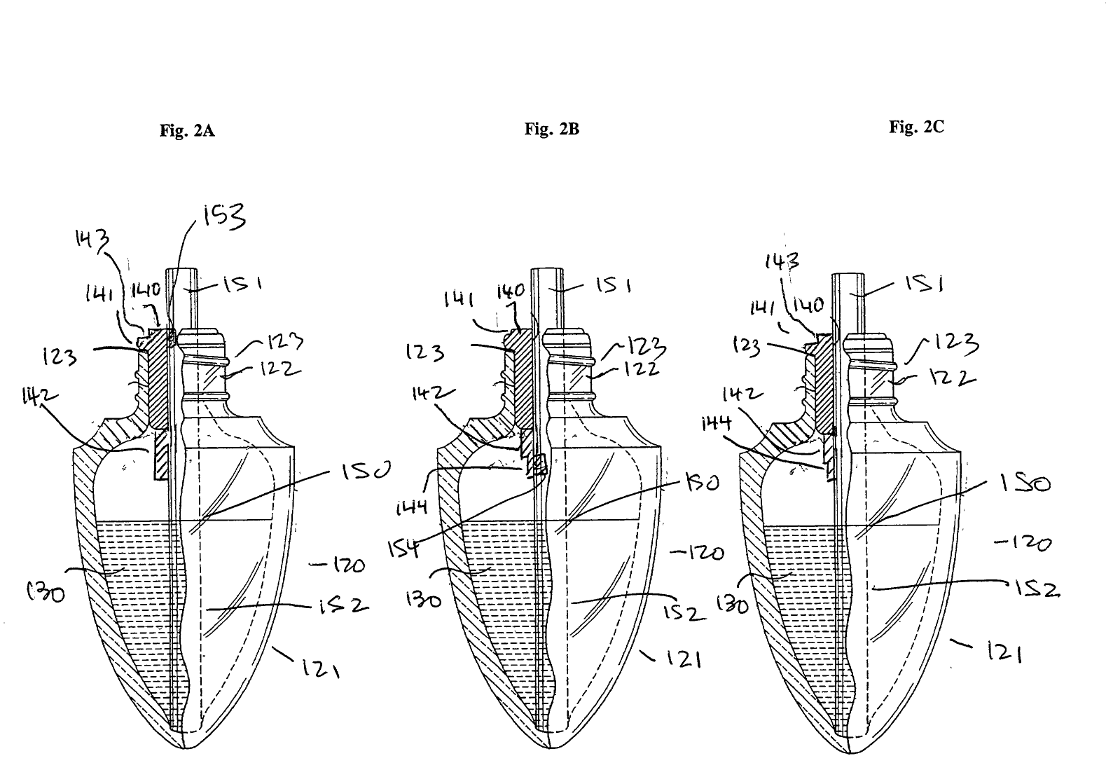

FIG. 1 is a schematic drawing of the air freshener device in a preferred embodiment of the present invention. FIG. 2 is a schematic drawing of three preferred embodiments of the invention illustrating the container, retaining ring, and wick of the air freshener device. In FIG. 1, the air freshener device is depicted generally as 100 and comprises a multi-part plastic vaporizer housing 110 having a socket portion 111. In FIGS. 1 and 2, a container 120 holds a liquid 130 to be evaporated by heat. Container 120 is preferably made of glass but any material which is compatible with the liquid 130 to be evaporated can be used as the container 120. The container 120 has a body 121 for storing the liquid 130 and a neck 122 connected to the body 121. The neck 122 engages the socket portion 111 of the housing 110 thereby supporting the container 120 on the housing 110. Socket portion 111 includes a threaded socket 112 for receiving the upper threaded neck end 123 of container 120 that contains liquid 130. Preferably, threaded socket 112 and upper threaded neck end 123 employ reverse screw threads. The neck 122 has a passage 123 therethrough. A retaining ring 140 is fixed in the passage 123 of the neck 122 of the container 120. A wick 150 has an upper portion 151 which extends through the retaining ring 140 and a lower portion 152 which extends into the body 121 of the container 120. The lower portion of the wick 152 absorbs the liquid 130 from the container 120 and moves the liquid 130 from the lower portion 152 into the upper portion 151 of the wick by capillary action. Wick 150 may be cotton, cellulose, polyester, or any other known wick material. Heating means 160 having electric plug blades 161 and a plug portion 162 in the housing 110 heats the upper portion of the wick 151, which is cylindrical in shape, above the neck 122 of the body 121 to evaporate the liquid 130 from the wick 151. The heating means 160 with plug portion 162 and electric blade means 161 is plugged into an electric wall socket for powering the heating means 160 and for supporting the air freshener device 100 on the wall. The heating means 160 is a tangential heater.

Passage 123 extends through the center of the retaining ring 140 and has a diameter slightly less than the outer diameter of the flexible fibrous wick 150 so that the upper portion of the wick 151 can extend through and be firmly held in passage 123 with the upper end of the wick 151 extending above the neck so that it can be heated by the heater assembly 160.

Preferably, threaded socket 112 and upper threaded neck end 123 employ reverse screw threads to minimize misuse of the product. With reverse threads, common forward screw thread containers cannot be used in the air freshener device. Reverse threads prevent a consumer from using volatile and flammable colognes or make-shift wicks in the device which would be dangerous. A typical container cannot be attached to the housing of the invention. The use of reverse screw threads means that container 120, when viewed from above, is turned clock-wise to tighten it into the vaporizer housing 110 and counter-clock-wise to loosen and remove it.

The container 120 of the air freshener device 100 may have a decorative body which simulates an item such as a bunch of fruit and is visible below the housing 110. The air freshener device 100 may further comprise a flange 170 extending upwardly from the housing 110 and simulating a further item which is consistent with the item simulated by the container 120. The flange 170 may mimic the leaves of a plant, the petals of a flower, or other shape. This outwardly extending flange 170 helps dissipate the evaporated air freshener, and decorates the device as it rests near a wall, supported by a wall socket. The flange 170 may also include a perforated dome and an annular recess 180. The socket portion 111 of the housing 110 may include a rim for engagement by the annular recess to hold the dome 180 and flange 170 to the housing 110.

FIG. 2 is a schematic drawing of three preferred embodiments of the invention illustrating the container, retaining ring, and wick of the air freshener device. In FIGS. 2A, 2B, and 2C, the retaining ring 140 has a higher cylindrical portion which forms a flange 141 on the top of the neck 122 of the container 120, and which engages the container by press fit, and a lower cylindrical portion 142. In FIG. 2A, the higher cylindrical portion 141 of the retaining ring 140 is crimped at 143 forming pressure point 153 on wick 151 by an amount which precludes upward extraction of the wick 151 from the retaining ring 140. In FIG. 2B, the lower cylindrical portion 142 of the retaining ring 140 is crimped at 144 forming pressure point 154 on wick 152 by an amount which precludes upward extraction of the wick 151 from the retaining ring 140. In FIG. 2C, both the higher 141 and the lower 142 cylindrical portions of the retaining ring 140 are crimped at 143 and 144 forming pressure points 153 and 154 on the wick by an amount which precludes upward extraction of the wick 150 from the retaining ring 140. The retaining ring 140 may also be glued to the neck 122 of the container 120. Retaining ring 140 may be made of polypropylene or other high strength synthetic material such as nylon.

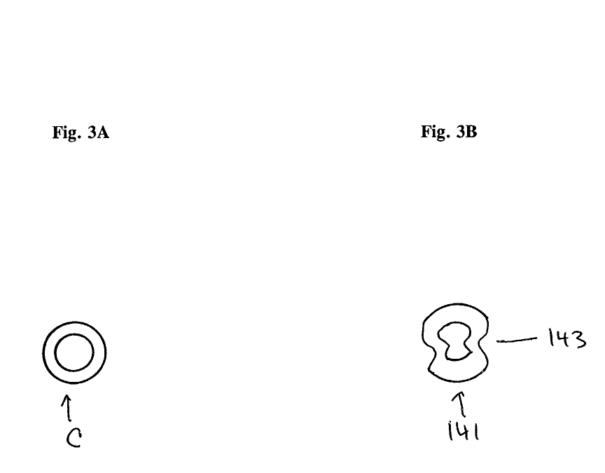

FIG. 3 is a schematic drawing comparing a top view of a conventional retaining ring for an air freshener device versus the crimped retaining ring of the present invention. FIG. 3A is a schematic drawing of a top view of a conventional retaining ring C for an air freshener device. FIG. 3B is a schematic drawing of a top view of the crimped retaining ring 140 of the present invention for an air freshener device. In FIG. 3B, the higher cylindrical portion 141 of the retaining ring 140 is crimped at 143 forming pressure point 153 on wick 151 by an amount which precludes upward extraction of the wick 151 from the retaining ring 140.

Retaining ring 140 may be crimped by any conventional method, such as with pliers or the like. Retaining ring 140 is pinched or pressed together in order to apply pressure and seal or unite retaining ring 140 with wick 150. The degree of crimping of retaining ring 140 at 143 or 144 to apply pressure, and engage, wick 150 at pressure point 153 or 154 is an amount sufficient to preclude extraction of wick 150 upwardly through retaining ring 140. Preferably, the degree of crimping of retaining ring 140 at 143 or 144 is such to require a pulling force of at least 5 pounds, preferably 10 pounds, and more preferably 15 pounds to extract wick 150 from container 120. This degree of crimping of retaining ring 140 effectively precludes extraction by a child and even some adults. This degree of crimping also makes it difficult to refill the container 120 with inappropriate liquids.

The heating means in the present invention is a tangential heater. Tangential heater assemblies are disclosed in U.S. Pat. No. 5,222,186, which disclosure is incorporated herein by reference. Tangential heater assemblies comprise a tangential heater which is surrounded by a ceramic potting material body. The tangential heater rests in one recess in the body and the body also contains a passage or opening for receiving the upper end of a wick. The opening may be slightly larger than the outer diameter of the wick to allow easy insertion of a new wick when the liquid in the container has been depleted. The upper end of the wick is automatically inserted into the opening when the neck is threaded into the socket.

The invention being thus described, it will be obvious that the same may be varied in many ways. Such variations are not to be regarded as a departure from the spirit and scope of the invention and all such modifications are intended to be included within the scope of the following claims.

We claim

1. An air freshener device comprising:
(a) a housing having a socket portion;
(b) a container for holding a liquid to be evaporated by heat, the container having a body for storing the liquid and a neck connected to the body, the neck engaging the socket portion of the housing thereby supporting the container on the housing, the neck having a passage therethrough;
(c) a retaining ring fixed in the passage of the neck of the container;
(d) a wick having an upper portion extending through the retaining ring and a lower portion extending into the body of the container, the lower portion of the wick absorbing the liquid from the container and moving the liquid from the lower portion into the upper portion of the wick by capillary action; and
(e) heating means in the housing for heating the upper portion of the wick above the neck of the body to evaporate the liquid from the wick;
wherein the retaining ring is crimped by an amount which precludes upward extraction of the wick from the retaining ring.

2. The device according to claim 1, wherein the neck of the container engages the socket portion of the housing with reverse screw threads.

3. The device according to claim 1, wherein the container has a decorative body which simulates an item and is visible below the housing.

4. The device according to claim 1, wherein the retaining ring has a higher cylindrical portion which forms a flange on the top of the neck of the container, and which engages the container by press fit, and a lower cylindrical portion.

5. The device according to claim 4, wherein the higher cylindrical portion of the retaining ring is crimped by an amount which precludes upward extraction of the wick from the retaining ring.

6. The device according to claim 4, wherein the lower cylindrical portion of the retaining ring is crimped by an amount which precludes upward extraction of the wick from the retaining ring.

7. The device according to claim 4, wherein both the higher and the lower cylindrical portions of the retaining ring are crimped by an amount which precludes upward extraction of the wick from the retaining ring.

8. The device according to claim 4, wherein the retaining ring is glued to the neck of the container.

9. The device according to claim 1, wherein the heating means includes a plug portion and electric blade means for being plugged into an electric wall socket for powering the heating means and for supporting the device on the wall.

10. The device according to claim 1, wherein the heating means is a tangential heater in the socket portion of the housing.

11. The device according to claim 3, further comprising a flange extending upwardly from the housing and simulating a further item which is consistent with the item simulated by the container.

12. The device according to claim 11, wherein the flange includes a perforated dome and an annular recess, the socket portion of the housing including a rim for engagement by the annular recess to hold the dome and flange to the housing.

13. An air freshener device comprising:
(a) a housing having a socket portion;
(b) a container for holding a liquid to be evaporated by heat, the container having a body for storing the liquid and a neck connected to the body, the neck engaging the socket portion of the housing with reverse screw threads thereby supporting the container on the housing, the neck having a passage therethrough;
(c) a retaining ring fixed in the passage of the neck of the container, the retaining ring having a higher cylindrical portion which forms a flange on the top of the neck of the container, and which engages the container by press fit, and a lower cylindrical portion;
(d) a wick having an upper portion extending through the retaining ring and a lower portion extending into the body of the container, the lower portion of the wick absorbing the liquid from the container and moving the liquid from the lower portion into the upper portion of the wick by capillary action; and
(e) heating means in the housing for heating the upper portion of the wick above the neck of the body to evaporate the liquid from the wick, the heating means including a plug portion and electric blade means for being plugged into an electric wall socket for powering the heating means and for supporting the device on the wall;
wherein the retaining ring is crimped by an amount which precludes upward extraction of the wick from the retaining ring.

14. The device according to claim 13, wherein the higher cylindrical portion of the retaining ring is crimped by an amount which precludes upward extraction of the wick from the retaining ring.

15. The device according to claim 13, wherein the lower cylindrical portion of the retaining ring is crimped by an amount which precludes upward extraction of the wick from the retaining ring.

16. The device according to claim 13, wherein both the higher and the lower cylindrical portions of the retaining ring are crimped by an amount which precludes upward extraction of the wick from the retaining ring.

17. The device according to claim 13, wherein the retaining ring is glued to the neck of the container.

18. The device according to claim 13, wherein the container has a decorative body which simulates an item and is visible below the housing.

19. The device according to claim 18, further comprising a flange extending upwardly from the housing and simulating a further item which is consistent with the item simulated by the container.

20. The device according to claim 13, wherein the heating means is a tangential heater in the socket portion of the housing.
